(19) United States
(12) Patent Application Publication
(10) Pub. No.: US 2019/0192543 A1
(43) Pub. Date: Jun. 27, 2019

(54) NOVEL USE

(71) Applicant: IDOGEN AB, Lund (SE)

(72) Inventors: Peter ERICSSON, Lund (SE); Hans Olov SJÖGREN, Lund (SE)

(73) Assignee: IDOGEN AB, Lund (SE)

(21) Appl. No.: 16/328,493

(22) PCT Filed: Aug. 25, 2017
(86) PCT No.: PCT/EP2017/071407

§ 371 Date: Feb. 26, 2019

(30) Foreign Application Priority Data
Aug. 26, 2016 (GB) 1614590.6

(51) Int. Cl. A61K 31/708; A61P 37/08; A61P 37/06; A61K 31/166; A61K 31/7068; A61K 31/165; A61K 31/436; A61K 9/00

(52) U.S. Cl. CPC A61K 31/708; A61P 37/08; A61P 37/06; A61K 9/0019; A61K 31/7068; A61K 31/165; A61K 31/436; A61K 31/166

(57) ABSTRACT

The present invention relates to ex vivo methods using guadecitabine, the compound of formula (I)

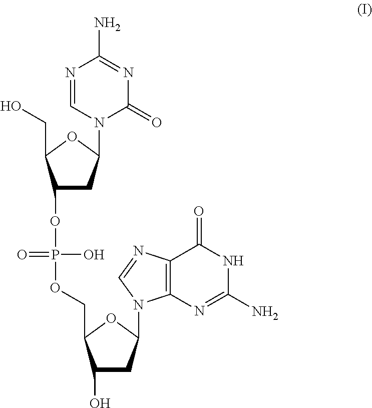 [SMILES: Nc1ncn([C@H]2C[C@@H](OP(=O)(O)OC[C@H]3O[C@@H](n4cnc5c(=O)[nH]c(N)nc54)C[C@H]3O)[C@@H](CO)O2)c(=O)n1]

and related uses.

### TECHNICAL FIELD

The invention relates to a compound and compositions comprising said compound for use in the treatment or prophylaxis of an immune related disease, disorder or condition, and to related methods.

### BACKGROUND TO THE INVENTION

IDO1, indoleamine 2,3-dioxygenase 1, is a heme-containing enzyme catalyzing the initial, rate-limiting step in tryptophan degradation that is known to have an important role in inducing immune tolerance in both experimental animals and in humans by inhibiting deleterious immune reactions. IDO gene expression is known to be induced in antigen presenting cells. When intracellularly expressed by antigen presenting cells such as dendritic cells, IDO1 functions as a natural immunoregulator by suppressing T cell responses which results in immune tolerance. Therefore control of IDO gene expression presents a possible mechanism for treating a variety of diseases in which the immune system is involved. For example, during mammalian pregnancy IDO1 plays a key role. IDO1 levels are increased during pregnancy and in this way it is essential for induction of immune tolerance against the foetus. Further, experimental inhibition of IDO1 in mice has been shown to result in rejection of foetuses expressing different MHC molecules than their mother.

Antigen presenting cells (APCs) are cells that display foreign peptide antigens complexed with major histocompatibility complexes (MHCs) on their surfaces; they process antigens and present them to T cells. APCs fall into two categories: professional and non-professional, the professional APC being those that express MHC class II molecules. There are four main types of professional APCs: dendritic cells, macrophages, certain B-cells and certain activated epithelial cells. Interaction of APCs with CD4+ T-cells occurs in the lymph nodes, to which APCs are directed through the effect of chemotaxis. Dendritic cells can be isolated and matured from a number of sources, such as bone marrow (particularly hematopoietic bone marrow progenitor cells) and blood (particularly peripheral blood mononuclear cells, of which some are immature and are expressing CD34). Dendritic cells may be obtained by differentiation of other less mature cells for example CD34+ hematopoietic stem cells or CD14+ monocytes. Depending on the circumstances, (for example, level of expression of co-stimulators such as CD80/86, CD40, or IDO1, PD-L1 or PD-L2 with suppressive activity), the interaction of dendritic cells and T cells in the lymph nodes can lead to an immune or a tolerant response. Thus, a key interaction involves DCs producing immunosuppressive cytokines (e.g. IL-10 and TGF-β), which induces the T cells to adopt a regulatory phenotype. The regulatory T cells (Treg cells) in turn do not only suppress the effector T cells or naïve T cells in their microenvironment, but they also induce immature DCs to differentiate to tolerogenic rather than immunogenic DCs thereby creating a tolerogenic loop response.

Guadecitabine, also known as SGI-110, is a dinucleotide comprising decitabine and deoxyguanosine and is also a DNA-hypomethylating agent that targets and reverses aberrant DNA hypermethylation (National Cancer Institute Drug Dictionary; Issa et al. 2015). It has been reported that guadecitabine-mediated demethylation of DNA upregulates tumour-associated antigens, may sensitise tumour cells to other anti-cancer agents and may resensitise resistant cancer cells to chemotherapeutics (Astex Pharmaceuticals; Clinical Pipeline). Guadecitabine delivers decitabine in vivo to rapidly dividing cancer cells and therefore represents a novel approach in the treatment of cancer. Guadecitabine has been found to be particularly effective in the treatment of acute myelogenous leukemia (AML) or myelodysplastic syndromes (MDS) (Issa et al. 2015) and phase I and II clinical trials are presently ongoing.

WO2008/147283 discloses the use of zebularine for the manufacturing of a medicament for the treatment of an auto-immune disorder or disease or immune rejection of transplants or gene therapeutically modified cells, wherein the treatment induces IDO.

WO2012/087234 discloses a composition comprising at least two compounds each of which induce indolamine 2,3-dioxygenase (IDO), for the treatment of an autoimmune disorder or disease or suffering from immune rejection of organs, tissues, normal cells or gene therapeutically modified cells, wherein said IDO inducers have different mechanisms of action and give rise to a synergistic effect on the IDO level. It also discloses a method of inducing IDO in a cell culture comprising the steps of (a) providing isolated cells in a suitable medium, (b) adding such a composition which comprises at least two compounds each of which induce IDO, (c) incubating said isolated cells with the composition and (d) obtaining a cell culture in which IDO is induced. It also discloses a method of treating a mammal having an autoimmune disorder or disease or suffering from immune rejection of organs, tissues, normal cells or gene therapeutically modified cells, wherein the treatment induces IDO, comprising firstly a treatment ex vivo of cells derived from the treated mammal or from another mammal, with a therapeutically effective amount of such a composition in the presence of one or more antigens associated with a condition being treated, followed by the transfer of treated cells to the mammal being treated.

### SUMMARY OF THE INVENTION

The inventors have surprisingly discovered that guadecitabine is a potent inducer of IDO in cells in which IDO is capable of being induced, for example THP-1 cells. This discovery makes possible important new treatment opportunities involving guadecitabine in circumstances where IDO induction would be beneficial, particularly immunotherapy.

Thus, the present invention provides a method of inducing IDO expression in a culture of antigen presenting cells obtained or derived from a subject who is suffering from or susceptible to an immune related disease, disorder or condition comprising the steps of:

(a) isolating antigen presenting cells or other cells which are capable of being matured or differentiated into antigen presenting cells, said antigen presenting cells being capable of IDO expression, from blood or bone marrow of the subject;

(b) culturing in vitro said antigen presenting cells or antigen presenting cells obtained by maturation or differentiation of said other cells in the presence of guadecitabine or a pharmaceutically acceptable salt thereof, and optionally one or more antigens associated with the immune related disease, disorder or condition, whereby IDO expression is induced in said antigen presenting cells.

Aspects of the invention involve culturing of CD4+ T cells isolated from the subject. Thus the present invention further provides such a method comprising the steps of:

(a) isolating antigen presenting cells or other cells which are capable of being matured or differentiated into antigen presenting cells, said antigen presenting cells being capable of IDO expression, and CD4+ T-cells which are capable of being differentiated into Treg cells, from the blood or bone marrow of the subject;

(b) co-culturing in vitro said antigen presenting cells or antigen presenting cells obtained by maturation or differentiation of said other cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells.

The present invention further provides a method of inducing tolerance in a subject who is suffering from or susceptible to an immune related disease, disorder or condition comprising the steps of:

(a) isolating antigen presenting cells or other cells which are capable of being matured or differentiated into antigen presenting cells, said antigen presenting cells being capable of IDO expression, from blood or bone marrow of the subject;

(b) culturing in vitro said antigen presenting cells or antigen presenting cells obtained by maturation or differentiation of said other cells in the presence of guadecitabine or a pharmaceutically acceptable salt thereof, and optionally one or more antigens associated with the immune related disease, disorder or condition, whereby IDO expression is induced in said antigen presenting cells;

(c) after IDO expression has been induced in said antigen presenting cells, transferring said antigen presenting cells back to the subject thereby to establish immune tolerance to the one or more antigens associated with the immune related disease, disorder or condition.

The present invention yet further provides a method of inducing tolerance in a subject who is suffering from or susceptible to an immune related disease, disorder or condition comprising the steps of:

(a) isolating antigen presenting cells or other cells which are capable of being matured or differentiated into antigen presenting cells, said antigen presenting cells being capable of IDO expression, and CD4+ T-cells which are capable of being differentiated into Treg cells, from the blood or bone marrow of the subject;

(b) co-culturing in vitro said antigen presenting cells or antigen presenting cells obtained by maturation or differentiation of said other cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells;

(c) after IDO expression has been induced in said antigen presenting cells and CD4+ T-cells have been differentiated into Treg cells, transferring the antigen presenting cells in which IDO expression has been induced or the Treg cells or both the antigen presenting cells in which IDO expression has been induced and the Treg cells back to the subject thereby to establish immune tolerance to the one or more antigens associated with the immune related disease, disorder or condition.

### BRIEF DESCRIPTION OF THE DRAWINGS

FIG. 1: Induction of IDO1 expression in THP-1 cells by guadecitabine.

### DETAILED DESCRIPTION OF THE INVENTION

### Definitions

The term “indolamine dioxygenase” or “IDO” as used herein means IDO1 (indoleamine 2,3-dioxygenase, EC 1.13.11.52) or IDO2 (indoleamine-pyrrole 2,3 dioxygenase-like 1, EC 1.13.11.-) these being two different proteins that can catabolize tryptophan, and can be expressed by APCs.

“Guadecitabine”, also known as SGI-110, means the compound of formula (I) below:

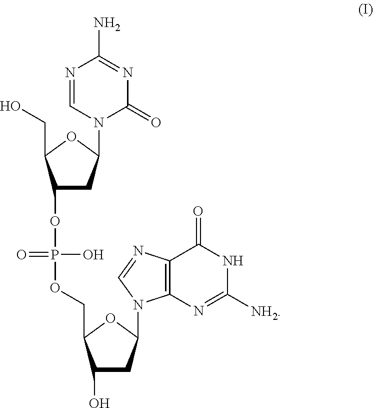 [SMILES: Nc1ncn([C@H]2C[C@@H](OP(=O)(O)OC[C@H]3O[C@@H](n4cnc5c(=O)[nH]c(N)nc54)C[C@H]3O)[C@@H](CO)O2)c(=O)n1]

“Immune related disease, disorder or condition” means any disease, disorder or condition in which a pathological immune response is observed.

“Immune tolerance” means the lack of response to antigens (self- or foreign-antigens) and includes natural tolerance or induced tolerance (i.e. deliberate manipulation of the immune system).

“Self-antigen” means any molecule or chemical group of an organism which acts as an antigen in inducing a T effector lymphocyte response or antibody formation in another organism but to which the healthy immune system of the parent organism is tolerant. Under certain circumstances, for example, when a subject is suffering from or is susceptible to an autoimmune disease, the parent organism is not tolerant to the self-antigen and a specific adaptive immune response is mounted against self-antigens.

“Exogenous therapeutic agent” means any therapeutic agent for treatment of a subject that originates from outside the subject.

The term “co-culturing” means culturing two (or more) cell types in the presence of each other.

It will be appreciated that in aspects of the invention guadecitabine may be employed in the form of a pharmaceutically acceptable salt. Likewise other substances to be used in combination with guadecitabine in different embodiments as referred to herein may also be employed in the form of a pharmaceutically acceptable salt. Suitable pharmaceutically acceptable salts will be apparent to those skilled in the art. Pharmaceutically acceptable salts include those described by Berge et al., 1977. Such pharmaceutically acceptable salts include acid addition salts formed with inorganic acids e.g. hydrochloric, hydrobromic, sulphuric, nitric or phosphoric acid and organic acids e.g. succinic, maleic, acetic, fumaric, citric, tartaric, benzoic, p-toluenesulfonic, methanesulfonic or naphthalenesulfonic acid.

Ex Vivo Methods of IDO Induction

Guadecitabine or a pharmaceutically acceptable salt thereof, or pharmaceutical composition comprising guadecitabine or a pharmaceutically acceptable salt thereof, may be employed in an ex vivo method of induction of IDO expression.

Suitably, guadecitabine or a pharmaceutically acceptable salt thereof is employed as the sole active ingredient in an ex vivo method of induction of IDO expression.

In one embodiment the present invention provides a method of inducing IDO expression in a culture of antigen presenting cells obtained or derived from a subject who is suffering from or susceptible to an immune related disease, disorder or condition comprising the steps of:

(a) isolating antigen presenting cells, said antigen presenting cells being capable of IDO expression, from blood or bone marrow of the subject;

(b) culturing in vitro said antigen presenting cells in the presence of guadecitabine or a pharmaceutically acceptable salt thereof, and optionally one or more antigens associated with the immune related disease, disorder or condition, whereby IDO expression is induced in said antigen presenting cells.

Suitable antigen presenting cells are disclosed elsewhere herein and include especially dendritic cells. Antigen presenting cells are most suitably derived from blood of the subject.

In a second embodiment the present invention also provides a method of inducing IDO expression in a culture of antigen presenting cells obtained or derived from a subject who is suffering from or susceptible to an immune related disease, disorder or condition comprising the steps of:

(a) isolating cells which are capable of being matured or differentiated into antigen presenting cells, said antigen presenting cells being capable of IDO expression, from blood or bone marrow of the subject;

(b) culturing in vitro antigen presenting cells obtained by maturation or differentiation of said cells which are capable of being matured or differentiated into antigen presenting cells in the presence of guadecitabine or a pharmaceutically acceptable salt thereof, and optionally one or more antigens associated with the immune related disease, disorder or condition, whereby IDO expression is induced in said antigen presenting cells.

Cells which are capable of being matured or differentiated into antigen presenting cells are disclosed elsewhere herein and include especially CD14+ monocytes and CD34+ hematopoietic stem cells. Such cells are most suitably derived from blood (e.g. peripheral blood) of the subject.

In a further embodiment the present invention provides a method of inducing IDO expression in a culture of antigen presenting cells obtained or derived from a subject who is suffering from or susceptible to an immune related disease, disorder or condition comprising the steps of:

(a) isolating antigen presenting cells, said antigen presenting cells being capable of IDO expression, and CD4+ T-cells which are capable of being differentiated into Treg cells, from the blood or bone marrow of the subject;

(b) co-culturing in vitro said antigen presenting cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells.

In a further embodiment the present invention provides a method of inducing IDO expression in a culture of antigen presenting cells obtained or derived from a subject who is suffering from or susceptible to an immune related disease, disorder or condition comprising the steps of:

(a) isolating cells which are capable of being matured or differentiated into antigen presenting cells, said antigen presenting cells being capable of IDO expression, and CD4+ T-cells which are capable of being differentiated into Treg cells, from the blood or bone marrow of the subject;

(b) co-culturing in vitro antigen presenting cells obtained by maturation or differentiation of said cells which are capable of being matured or differentiated into antigen presenting cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells.

As noted in the Background section, antigen presenting cells such as dendritic cells and T cells can interact with each other and a key interaction involves DCs expressing IDO and producing immunosuppressive cytokines (e.g. IL-10 and TGF-β) which induces the T cells to adopt a regulatory phenotype. Without being limited by theory, it is believed that by functioning as a signal transducing protein for the TGF-beta receptor, IDO especially IDO1, induced in DCs by guadecitabine, further enhances the TGF-beta and the IDO expression of the dendritic cells. The regulatory T cells in their turn can also induce immature DCs to differentiate to tolerogenic rather than immunogenic DCs thereby creating a tolerogenic loop response. Hence the co-culturing of antigen presenting cells or other cells which are capable of being matured or differentiated into antigen presenting cells together with CD4+ T-cells in the culture is expected to lead to beneficial expansion of the tolerogenic cell population in the cell culture. In addition, without being limited by theory, the guadecitabine may have a direct effect on the T-cells by inducing differentiation to Treg cells via demethylation of FoxP3 and CTLA4 thus leading to activation of these two genes for Treg regulating function. In summary, particular benefits are expected to arise from the presence in the culture of antigen presenting cells, CD4+ T-cells and guadecitabine.

In a further embodiment the present invention provides a method of inducing tolerance in a subject who is suffering from or susceptible to an immune related disease, disorder or condition comprising the steps of:

(a) isolating antigen presenting cells or cells which are capable of being matured or differentiated into antigen presenting cells, said antigen presenting cells being capable of IDO expression, from blood of the subject;

(b) culturing in vitro said antigen presenting cells in the presence of guadecitabine or a pharmaceutically acceptable salt thereof, and optionally one or more antigens associated with the immune related disease, disorder or condition, whereby IDO expression is induced in said antigen presenting cells;

(c) after IDO expression has been induced in said antigen presenting cells, transferring said cells back to the subject thereby to establish immune tolerance to the one or more antigens associated with the immune related disease, disorder or condition.

In a further embodiment the present invention provides a method of inducing tolerance in a subject who is suffering from or susceptible to an immune related disease, disorder or condition comprising the steps of:

(a) isolating antigen presenting cells or cells which are capable of being matured or differentiated into antigen presenting cells, said antigen presenting cells being capable of IDO expression, from blood of the subject;

(b) culturing in vitro antigen presenting cells obtained by maturation or differentiation of said cells which are capable of being matured or differentiated into antigen presenting cells in the presence of guadecitabine or a pharmaceutically acceptable salt thereof, and optionally one or more antigens associated with the immune related disease, disorder or condition, whereby IDO expression is induced in said antigen presenting cells;

(c) after IDO expression has been induced in said antigen presenting cells, transferring said cells back to the subject thereby to establish immune tolerance to the one or more antigens associated with the immune related disease, disorder or condition.

In a further embodiment the present invention provides a method of inducing tolerance in a subject who is suffering from or susceptible to an immune related disease, disorder or condition comprising the steps of:

(a) isolating antigen presenting cells, said antigen presenting cells being capable of IDO expression, and CD4+ T-cells which are capable of being differentiated into Treg cells, from the blood of the subject;

(b) co-culturing in vitro said antigen presenting cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells;

(c) after IDO expression has been induced in said antigen presenting cells and CD4+ T-cells have been differentiated into Treg cells, transferring either the antigen presenting cells in which IDO expression has been induced, or the Treg cells, or both the antigen presenting cells in which IDO expression has been induced and the Treg cells, back to the subject thereby to establish immune tolerance to the one or more antigens associated with the immune related disease, disorder or condition.

In a further embodiment the present invention provides a method of inducing tolerance in a subject who is suffering from or susceptible to an immune related disease, disorder or condition comprising the steps of:

(a) isolating cells which are capable of being matured or differentiated into antigen presenting cells, said antigen presenting cells being capable of IDO expression, and CD4+ T-cells which are capable of being differentiated into Treg cells, from the blood of the subject;

(b) co-culturing in vitro antigen presenting cells obtained by maturation or differentiation of said cells which are capable of being matured or differentiated into antigen presenting cells and CD4+ T-cells in the presence of guadecitabine or a pharmaceutically acceptable salt thereof, and optionally one or more antigens associated with the immune related disease, disorder or condition, whereby IDO expression is induced in said antigen presenting cells and whereby said CD4+ T-cells are differentiated into Treg cells;

(c) after IDO expression has been induced in said antigen presenting cells and CD4+ T-cells have been differentiated into Treg cells, transferring either the antigen presenting cells in which IDO expression has been induced, or the Treg cells, or both the antigen presenting cells in which IDO expression has been induced and the Treg cells, back to the subject thereby to establish immune tolerance to the one or more antigens associated with the immune related disease, disorder or condition.

There can be a therapeutic benefit to the subject in transferring into said subject just the antigen presenting cells. In vivo these will have a beneficial effect e.g. in inducing the production of Treg cells in vivo. Alternatively there can be a therapeutic benefit to the subject in transferring into said subject just the Treg cells which can have their direct immune regulating effect in vivo. Alternatively, both types of cells can be transferred into the subject. Where the cell types need to be separated, this can be done by conventional cell sorting techniques for example using magnetic beads coated with or linked to monoclonal antibodies against marker molecules presented on the cell surface (such as CD4, CD14, CD34, CD11c, CD1a or CD123).

Thus, in an embodiment, in step (c) either the antigen presenting cells in which IDO expression has been induced or the Treg cells are transferred back to the subject. Alternatively, in another embodiment, in step (c) both the antigen presenting cells in which IDO expression has been induced and the Treg cells are transferred back to the subject.

Antigen Presenting Cells (APCs)

APCs suitable for use in the present invention are APCs which are capable of expressing IDO, especially IDO1. APCs are cells capable of inducing a tolerogenic loop, for example by generating antigen specific regulatory T cells or B cells. In one embodiment, the APCs are dendritic cells (DCs), such as bone marrow-derived dendritic cells (BMDCs).

Other cells that are suitable for use in the present invention are cells which are capable of being matured or differentiated into antigen presenting cells such as dendritic cells. Such cells include stem cells such as CD34+ hematopoietic stem cells or CD14+ monocytes. Antigen presenting cells and cells which are capable of being matured or differentiated into antigen presenting cells such as CD34+ hematopoietic stem cells and CD14+ monocytes are suitably derived from peripheral blood. DCs that are capable of proliferating are preferred. The hematopoietic stem cells are readily expandable, but it also appears possible to induce DCs derived from peripheral blood CD14+ monocytes-to proliferate by appropriate treatment (see e.g. Langstein et al., 1999). Dendritic cells may be mature dendritic cells or immature dendritic cells. The dendritic cells may be dendritic cells that have been co-cultured with mesenchymal stem cells.

Antigens

In step (b) of the above ex vivo methods, one or more antigens may be (and suitably are) included in the culture. An antigen is any substance that causes the immune system to react e.g. by generating T-cells recognizing peptides derived from protein substances, and B-cells producing antibodies against the substance. The antigen will bear one or more epitopes.

In the case of the immune related disease, disorder or condition being an autoimmune disease or disorder, the one or more antigens comprise self-antigens e.g. antigens derived from collagen, cartilage or other tissues of the subject.

In the case of the immune related disease, disorder or condition being immune rejection of transplant of organs, tissues or cells, the one or more antigens comprise antigens derived from the transplant.

In the case of the immune related disease, disorder or condition being immune reactions to exogenous therapeutic agent, the antigen preparation corresponding to the exogenous therapeutic agent, for example a drug, may contain the exogenous therapeutic agent, for example a drug, in whole or part, said part comprising an epitope containing fragment thereof. Generally, a purified antigen will be used. Suitably, the antigen preparation corresponding to the exogenous therapeutic agent, for example a drug, resembles as closely as possible the exogenous therapeutic agent, for example a drug, which is being administered to the subject. In the case of FVIII, various recombinant drugs are available which are suitable for this purpose including the commercial products ReFacto AF, Helixate NexGen, Kogenate Bayer, Advate, NovoEight, Nuwiq, Beriate, Beriate P, Haemoctin, Hemofil, Monoclate-P, Octanate [LV], Optivate and Recombinate. In the case of FIX, various recombinant drugs are available which are suitable for this purpose including the commercial products Immunine, Mononine, Alprolix, Rixubis and Benefix.

Ex Vivo Methods of IDO Induction for Treatment of an Immune Related Disease, Disorder or Condition

The immune related disease, disorder or condition may be any one described herein, for example under the heading “Disease Indications”.

In one embodiment of the present invention, the subject is suffering from or susceptible to an autoimmune disease or disorder (as described below) and the one or more antigens comprise self-antigens e.g. antigens derived from collagen, cartilage or other tissues of the subject. In a particular embodiment of the invention, the subject is suffering from or susceptible to rheumatoid arthritis and the one or more antigens comprise antigens derived from collagen, cartilage or other tissues of the subject.

In a further embodiment, the subject is suffering from or susceptible to immune rejection of a transplant and the one or more antigens comprise antigens derived from the transplant. Suitably the transplant is transplant of an organ, tissue or cells (including normal cells or genetically modified cells). The organ, tissue or cells are as described below.

In a yet further embodiment, the subject is suffering from or susceptible to immune reaction to an exogenous therapeutic agent and the one or more antigens comprise antigens from the exogenous therapeutic agent. The exogenous therapeutic agent is as described below and thus may be a biological drug such as FVIII, Factor IX or an antibody and the immune reaction comprises the raising of antibodies thereto.

Ex Vivo Combination Methods of IDO Induction

In one embodiment, one or more additional active ingredients are employed in step (b) of the aforementioned ex vivo methods in addition to guadecitabine. Such active ingredients may be selected from metabolites of tryptophan; immunosuppressive reagents; aldehyde oxidase inhibitors; methotrexate; rapamycin; cyclophosphamide; antimetabolites; immunophilin-binding drugs; inhibitors of nucleotide synthesis; FTY720; lymphocyte depleting antibodies; non-depleting antibodies; anti-TNF antibodies; natalizumab; anti-CD154 antibodies; soluble cytokine receptors; soluble TNF receptors; and anakinra. Suitably the one or more additional active ingredient includes rapamycin.

In one embodiment one or more additional active ingredients which induce IDO are employed in step (b) and are selected from compounds such as cytidine analogues; histone deacetylase inhibitors; vitamin D3 analogues; interferons; toll-like receptor ligands; gonadotropine receptor signalling hormones; prostaglandine E2 analogues; IDO stabilizers; soluble CTLA4 conjugates; and glycocorticoids. More suitably, an additional active ingredient is a cytidine analogue, for example, zebularine or a pharmaceutically acceptable salt thereof. Alternatively, an additional active ingredient which induces IDO is an interferon, for example interferon gamma or interferon alpha or a pharmaceutically acceptable salt thereof. In one embodiment, the cytidine analogue is not zebularine or a pharmaceutically acceptable salt thereof.

In a further embodiment, the one or more additional active ingredients which induce IDO are selected from the group consisting of procainamide, decitabine, psammaplin A and azacytidine and pharmaceutically acceptable salts thereof.

In a further embodiment, one or more additional active ingredients are employed in step (b) and are other than procainamide, psammaplin A, azacytidine or decitabine, or pharmaceutically acceptable salts thereof.

In a yet further embodiment, guadecitabine may be administered with two additional active ingredients such as a cytidine analogue (for example zebularine or a pharmaceutically acceptable salt thereof) and an interferon such as interferon gamma or interferon alpha or a pharmaceutically acceptable salt thereof. Suitably, the cytidine analogue is not zebularine or a pharmaceutically acceptable salt thereof.

In one embodiment, step (b) of the above ex vivo methods further comprises stimulating the cultured antigen presenting cells with oligonucleotides.

Cell Cultures in which IDO Expression has been Induced

Cell cultures may be prepared in which IDO is induced ex vivo.

In an embodiment of the invention there is provided antigen presenting cells in which IDO expression has been induced, obtained or obtainable by the any of the methods of the present invention.

Suitably, the cell culture in which IDO expression has been induced comprises:
(a) isolated antigen presenting cells or antigen presenting cells obtained by maturation or differentiation of other isolated cells which are capable of being matured or differentiated into antigen presenting cells from blood or bone marrow of a subject; and
(b) guadecitabine or a pharmaceutically acceptable salt thereof.

Suitably, the cell culture in which IDO expression has been induced further comprises Treg cells obtained by differentiation in the cell culture of CD4+ T-cells isolated from blood or bone marrow of the subject.

Suitably, the cell culture in which IDO expression has been induced further comprises one or more antigens associated with the immune related disease, disorder or condition. Suitably the immune related disease, disorder or condition is selected from the group consisting of autoimmune diseases and disorders; immune rejection of transplants; and immune reactions to an exogenous therapeutic agent and are as defined below.

In one embodiment there are provided antigen presenting cells obtained or obtainable according to methods of the invention or isolated from a cell culture according to the invention for use in a method of treating a subject suffering from or susceptible to an immune related disease, disorder or condition whereby according to any of the methods of the present invention said cells are transferred back to said subject thereby to establish immune tolerance to the immune related disease, disorder or condition.

In another embodiment there are provided Treg cells isolated from a cell culture according to the invention for use in a method of treating a subject suffering from or susceptible to an immune related disease, disorder or condition whereby according to said method said cells are transferred back to said subject thereby to establish immune tolerance to the immune related disease, disorder or condition.

In another embodiment there are provided antigen presenting cells obtained or obtainable according to methods of the invention or isolated from a cell culture according to the invention in combination with Treg cells isolated from a cell culture according to the invention for use in a method of treating a subject suffering from or susceptible to an immune related disease, disorder or condition whereby according to said method said cells in combination are transferred back to said subject thereby to establish immune tolerance to the immune related disease, disorder or condition.

More Detailed Ex Vivo IDO Induction Methodology

1. Obtaining or Deriving APCs from a Subject

APCs such as DCs are first obtained from a subject for example a mammal, e.g. a human. One suitable method is to collect immature PBMCs by apheresis (optionally after mobilization of immature cells with standard doses of recombinant G-CSF). Another suitable method is to use Nycodenz centrifugation to separate low density monocytes after lyzing of the red blood cells. To obtain a population of proliferating DCs, immature CD34⁺ hematopoietic stem cells may be purified from PBMC using magnetic beads linked to anti-CD34 MAb (Dyna) Biotech, Oslo, Norway). CD34⁺ hematopoietic stem cell purity can be confirmed by flow cytometry. Purified CD34⁺ hematopoietic stem cells are typically differentiated into DCs by treatment with GM-CSF, SCF and IL-4. Thus, typically they are cultured at 1×10⁵ cells/mL in tissue culture flasks (Nunc, Roskilde, Denmark) in SCGM serum-free medium, CellGro serum-free medium or RPMI medium containing 10% autologous plasma, 2 mM l-glutamine (JHR), 20-100 ng/ml GM-CSF, 20-100 ng/mL SCF, 20-100 ng/mL, IL-4 (R&D Systems, Minneapolis, Minn., USA), optionally together with 100 ng/mL IL3, 5-20 ng/mL IL10 and 50 ng/ml Flt-3L (R&D Systems), 10 ng/mL TNF-alpha and/or 10 ng/mL IL1-beta. Cells may also be cultured with foetal bovine serum (FBS) or human serum AB. Cultures may be maintained at 37° C. in humidified 5% CO₂ atmosphere for 14 days. As cellular expansion occurs, cell culture medium and cytokines are replenished (e.g. approximately every 4 days).

In some circumstances it may be possible to isolate BMDCs from a bone of the subject. DC cell differentiation can be induced and cells expanded as described above for CD34⁺ hematopoietic stem cells.

CD14+ monocytes (e.g. from peripheral blood) are differentiated into DCs using a similar method to that described above. CD14 cells are purified using a similar method as described above, using anti-CD14 Mab and cultured in CellGro serum free or SCGM serum free for 2-5 days in GM-CSF and IL4. The cell culture is matured over two to three days with TNF-alpha, IL1-beta, PGE2 and/or IL-10.

2. Ex Vivo Exposure to Guadecitabine

At day 12-14 immature DCs may be exposed to guadecitabine, together with an antigen preparation e.g. corresponding to the tissue for transplant, self-antigen or exogenous therapeutic agent. The concentration of guadecitabine and the length of exposure can be selected for optimum IDO induction. A concentration of 0.1 microM-50 mM such as 0.1-30 microM e.g. 0.1-10 microM is exemplary. An exposure of 2-4 days is exemplary.

Guadecitabine or a pharmaceutically acceptable salt thereof and the antigen preparation can be added to the DCs together or separately. Antigens and guadecitabine or a pharmaceutically acceptable salt thereof may be added at the same time as maturation of immature cells such as CD34⁺ hematopoietic stem cells and CD14⁺ monocytes into DCs.

In order to achieve appropriate migration of the DCs to lymph nodes upon administering the cells into the patient, cells are typically treated for approximately 24 h with prostaglandin E2 (at concentration 1-10 μM) to induce required chemotactic receptors.

At day 15-17, DCs may be harvested, suspended in medium and stored frozen in liquid nitrogen until being released for use.

Typically, a quality control step will be performed prior to use. Quality control should include tests for viability, sterility and endotoxin and expression of IDO1. Additionally, expressions of PD-L1, PD-L2, the chemotactic receptor CCR7, level of expression of the co-stimulators CD80/CD86, CD40, and MHC-II are to be considered. Expression levels of these substances is an indicator that the DCs are tolerogenic.

3. Obtaining CD4+ Cells from a Subject, Co-Culture of APCs with CD4+ T-Cells in the Presence of Guadecitabine and Inducing Treg Cells In Vitro

Using anti-CD4 and anti-CD25 monoclonal antibodies attached to magnetic beads, CD4+CD25− T-cells are isolated by magnetic separation and co-cultured with APCs, or cells being capable of being differentiated into APCs particularly DCs, in CellGro serum free or SCGM serum free containing tryptophan restricted to 1-3 uM for 3-10 days in the presence of guadecitabine and IL-2 (2000 U/ml) and anti-CD3 beads to allow expansion of the generated CD4+ Treg cells.

Administration of Cells to the Subject

Cells may be administered back to the mammal by various routes e.g. intravenously, subcutaneously or intracutaneously. The medium containing the cells is suitably containing human albumin as a cell-protecting protein. Typically an amount of 3-10×10⁶ APCs/dose and/or 3-30×10⁶ Treg cells/dose in 1-10 doses at weekly to bi-weekly intervals is administered. The treatment can be extended until the desired tolerance is achieved.

Optionally, the subject may receive treatment with one or more other pharmaceutically active agents concurrently with the treatment with cells. Thus, concurrently with the treatment with cells the subject may receive treatment with rapamycin. As noted elsewhere herein, rapamycin may be expected to have a role in expanding the population of Treg cells in vivo

Ex Vivo Treatment of Transplants in which IDO is Induced

Transplants may be treated with guadecitabine or a pharmaceutically acceptable salt thereof prior to implantation of the transplant to a subject in need thereof, such that IDO is induced in the transplant prior to implantation thereby establishing immune tolerance in said subject post-implantation of the transplant.

Thus, in one embodiment of the invention, there is provided a method of inducing IDO in a transplant comprising the steps of:

(a) obtaining a transplant (for example, organ, tissue or cells) from a donor;

(b) administering ex vivo to said transplant an effective amount of guadecitabine or a pharmaceutically acceptable salt thereof such that IDO is induced in cells of the transplant.

In a second embodiment, the present invention further provides a method of inducing immune tolerance to a transplant in a subject who is to receive said transplant comprising the steps of:

(a) obtaining a transplant (for example, organ, tissue or cells) from a donor;

(b) administering ex vivo to said transplant an effective amount of guadecitabine or a pharmaceutically acceptable salt thereof such that IDO is induced in cells of the transplant;

(c) after IDO expression has been induced in said transplant, implanting the transplant in the subject in need thereof such that immune tolerance to the transplant in the subject is established.

In one embodiment, the transplant is an organ, tissue, or cells (including normal cells or genetically modified cells). Organ, tissue or cells may be any one of those described below. In a particular embodiment, the transplant is cells, in particular, cells derived from the patients' own stem cells differentiated in vitro to express molecules such as insulin or cells derived from the patients' own cells which have been genetically manipulated to express key molecules, wherein a specific immune response may be observed against the cell transplant. In patients with type I diabetes, for example, the specific immune response may be against antigens selective for beta-cells or insulin, proinsulin or preproinsulin. In one embodiment, the transplanted cells may be pancreatic insulin-producing beta-cells. Patients who receive cells which have been genetically manipulated to express key molecules may be unable to express the key molecules, for example, due to errors of metabolism.

The effective amount of guadecitabine or pharmaceutically acceptable salt will depend on the transplant. The effective amount of guadecitabine required to induce IDO may be between 0.1-50 microM such as 0.1-30 microM e.g. 0.1-10 microM.

In one embodiment, guadecitabine is employed in step (b) as the sole active ingredient.

In one embodiment, one or more additional active ingredients are employed in step (b) and are selected from metabolites of tryptophan; immunosuppressive reagents; aldehyde oxidase inhibitors; methotrexate; rapamycin; cyclophosphamide; antimetabolites; immunophilin-binding drugs; inhibitors of nucleotide synthesis; FTY720; lymphocyte depleting antibodies; non-depleting antibodies; anti-TNF antibodies; natalizumab; anti-CD154 antibodies; soluble cytokine receptors; soluble TNF receptors; and anakinra.

In one embodiment, one or more additional active ingredients which induce IDO are employed in step (b) and are selected from compounds such as cytidine analogues (e.g. zebularine or a pharmaceutically acceptable salt thereof); histone deacetylase inhibitors; vitamin D3 analogues; interferons (e.g. interferon gamma or interferon alpha or a pharmaceutically acceptable salt thereof); toll-like receptor ligands; gonadotropine receptor signalling hormones; prostaglandine E2 analogues; IDO stabilizers; soluble CTLA4 conjugates; and glycocorticoids. In one embodiment, the cytidine analogue is not zebularine or a pharmaceutically acceptable salt thereof.

Suitably, one or more additional active ingredients which induce IDO are employed in step (b) and are selected from the group consisting of procainamide, psammaplin A, decitabine and azacytidine and pharmaceutically acceptable salts thereof.

Suitably, one or more additional active ingredients are employed in step (b) and are other than procainamide, psammaplin A, decitabine or azacytidine, or pharmaceutically salts thereof.

In a yet further embodiment, guadecitabine may be employed in step (b) with two additional active ingredients such as a cytidine analogue (for example zebularine or a pharmaceutically acceptable salt thereof) and an interferon such as interferon gamma or interferon alpha or a pharmaceutically acceptable salt thereof. Suitably, the cytidine analogue is not zebularine or a pharmaceutically acceptable salt thereof.

In Vivo Uses

The invention also provides guadecitabine or a pharmaceutically acceptable salt thereof, for use in the prophylaxis or treatment of an immune related disease, disorder or condition.

The present invention further provides use of guadecitabine or a pharmaceutically acceptable salt thereof for the manufacture of a medicament for the prophylaxis or treatment of an immune related disease, disorder or condition.

The present invention also provides a method of treating an immune related disease, disorder or condition comprising administering to a subject in need thereof a therapeutically effective amount of guadecitabine or a pharmaceutically acceptable salt thereof.

Disease Indications

Guadecitabine, or a pharmaceutically acceptable salt thereof, may be used as a medicament, in particular for the prophylaxis or treatment of an immune related disease, disorder or condition.

Suitably, the immune related disease, disorder or condition is selected from the group consisting of autoimmune diseases and disorders; immune rejection of transplants; and immune reactions to an exogenous therapeutic agent.

Autoimmune Diseases or Disorders

In one embodiment, the immune related disease, disorder or condition is an autoimmune disease or disorder.

Suitably the autoimmune diseases or disorders are selected from the group consisting of Achlorhydria, Acute haemorrhagic leukoencephalitis, Addison's Disease, Alopecia Areata, Anemia, Ankylosing Spondylitis, Anti-Glomerular Basement Membrane Disease, Antiphospholipid Syndrome, Aplastic Anemia, Atopic Allergy, Autoimmune Atrophic Gastritis, Autoimmune Hearing Loss, Autoimmune hemolytic anemia, Autoimmune Hepatitis, Autoimmune hypoparathyroidism, Autoimmune hypophysitis, Autoimmune Lymphoproliferative, Autoimmune Myocarditis, Autoimmune oophoritis, Autoimmune orchitis, Autoimmune Polyendocrinopathy-Candidiasis-Ectodermal-Dystrophy, Autoimmune Polyendocrinopathy, Autoimmune Syndrome Type II, Behcet Syndrome, Celiac Disease, Chagas Disease, Chronic Active Hepatitis, Chronic Inflammatory Demyelinating Polyneuropathy, Chronic lymphocytic thyroiditis, Churg-Strauss Syndrome, Crohn's disease, Cryoglobulinemia, Cushing Syndrome, Dermatitis Herpetiformis, Dermatomyositis, Diabetes Mellitus type 1, Diffuse Cerebral Sclerosis of Schilder, Experimental Autoimmune Encephalomyelitis (EAE), Epidermolysis Bullosa Acquisita, Erythematosis, Experimental Autoimmune Neuritis, Felty's Syndrome, Glomerulonephritis, Glomerulonephritis Membranous, Goodpasture Syndrome, Graves' Disease, Guillain-Barre Syndrome, Hamman-Rich syndrome, Idiopathic Thrombocytopenic Purpura, Inflammatory Bowel Diseases, Insulin resistance-type B, Lambert-Eaton Myasthenic Syndrome, Lens-induced uveitis, Lichen Sclerosus et Atrophicus, Lymphopenia, Meniere's Disease, Mixed Connective Tissue Disease, Mooren's ulcer, Mucocutaneous Lymph Node Syndrome, Multiple Sclerosis, Myasthenia Gravis, Myelitis Transverse, Myocarditis, Narcolepsy, Neuromyelitis Optica, Oculovestibular auditory syndrome, Ophthalmia Sympathetic, Opsoclonus-Myoclonus Syndrome, Pancreatitis, Pemphigoid Bullous, Pemphigus foliaceous, Pemphigus Vulgaris, Polyarteritis Nodosa, Polymyalgia Rheumatica, Polyradiculoneuropathy, Primary biliary cirrhosis, Psoriasis, Raynauds, Reiter Disease, Relapsing Polychondritis, Rheumatic Fever, Rheumatoid Arthritis, Sarcoidosis, Scleroderma, Sclerosing Cholangitis, Sjögren's Syndrome, Stiff-Person Syndrome, Adult-Onset Still's Disease, Takayasu's Arteritis, Temporal Arteritis, Thyrotoxicosis, Type B Insulin Resistance, Ulcerative Colitis, Uveomeningoencephalitic Syndrome, Vitiligo and Wegener's Granulomatosis.

Suitably, the autoimmune disease is selected from the group consisting of Diabetes Mellitus Type 1, Rheumatoid Arthritis, Chronic lymphocytic thyroiditis, Multiple Sclerosis and Ulcerative Colitis.

In addition, diseases that can partly be involved with autoimmune reactivity are arteriosclerosis, Parkinson's disease, and Alzheimer's disease.

Immune Rejection of Transplants

In another embodiment, the immune related disease, disorder or condition is immune rejection of transplants.

Suitably the transplant is an organ, tissue or cells (including normal cells or genetically modified cells). Organ transplants include transplant of kidney, liver, heart, lung, pancreas, intestines and thymus (or part of said organ). In one embodiment, the transplant is an organ, such as kidney, liver, heart or lung.

Tissue transplants include transplant of bones, tendons (collectively, musculoskeletal grafts), cornea, skin, tendon, heart valves, nerves and veins. In one embodiment, the tissue is nerve tissue, for example fetal ventral mesencephalic tissue. Typically the transplant is an allograft. Alternatively it might be a xenograft.

Cell transplants include cells derived from the subject's own stem cells differentiated in vitro to express molecules such as insulin or cells derived from the subject's own cells which have been genetically manipulated to express molecules that the subject is unable to express e.g. due to error of metabolism or genetic defect. In one embodiment, the transplanted cells may be pancreatic insulin-producing beta-cells. In another embodiment, the cell is a nerve cell, for example a neuron.

Immune Rejection of an Exogenous Therapeutic Agent

In another embodiment, the immune related disease, disorder or condition is an immune reaction to an exogenous therapeutic agent.

The exogenous therapeutic agent may be any drug capable of generating an immune response for example any biological drug e.g. a protein drug. The immune reaction typically includes the raising of antibodies thereto.

Example protein drugs include blood factors (including FVIII or Factor IX), hormones (including insulin and EPO), growth factors (including EGF, IGF, KGF, HGF and FGF), cytokines (e.g. interleukins), enzymes and the like. In one embodiment of the invention, the drug is FVIII. In another embodiment of the invention, the drug is Factor IX.

Biological drugs may contain polysaccharide components.

Further example biological drugs include engineered proteins (such as fusion and chimeric proteins) and recombinant proteins. In some embodiments of the invention the drug may be an antibody. The drug may, for example, be a monoclonal antibody, such as a humanized or fully human monoclonal antibody. The drug may also be a protein construct comprising fragments of immunoglobulin. The term antibody includes antibody-drug conjugates and antibody-nanoparticle conjugates as well as PEGylated analogues. In some embodiments the antibody may be a domain antibody including a single light chain antibody or VHH. The drug may consist of an intact light chain immunoglobulin, or a fragment thereof which comprises at least a variable domain and at least part of the light chain constant region or a ScFv. The drug may be free of heavy chain immunoglobulins. Antibodies include anti-TNF-α monoclonal antibodies, for example, REMICADE™ (infliximab), ENBREL™ (etanercept), HUMIRA™ (adalimumab), CIMZIA® (certolizumab pegol), SIMPONI® (golimumab; CNTO 148) and anti-VEGF antibodies.

Methods of the invention are suitable for the treatment of subjects who develop an immune reaction to a drug including the raising of anti-drug antibodies in a number of drug treatment situations, such as bleeding disorders (haemophilia A and B; respectively deficiency of FVIII and Factor IX), growth factor deficiency (deficiency of EGF, IGF, KGF, HGF, FGF etc), hormone deficiency (deficiency of insulin, EPO), enzyme replacement therapy and inflammatory and auto-immune disorders (anti-TNF-α monoclonal antibodies).

An extensive list of biological protein therapeutics in clinical development and approved products are disclosed in the 2013 PhRMA report “Biologics”-http://www.phrma.org/sites/default/files/pdf/biologics2013.pdf—which gives details of 907 biologics targeting more than 100 diseases. This document is incorporated by reference in its entirety. It is considered that the present invention may be used against these as well as other biological therapeutics where an immune response is developed in the treated subject.

Combination Therapy

In one embodiment, guadecitabine is administered as the sole active ingredient.

In another embodiment, guadecitabine may be administered in combination with one or more additional active ingredients.

Suitably the one or more additional active ingredients are selected from metabolites of tryptophan; immunosuppressive reagents; aldehyde oxidase inhibitors; methotrexate; rapamycin; cyclophosphamide; antimetabolites; immunophilin-binding drugs; inhibitors of nucleotide synthesis; FTY720; lymphocyte depleting antibodies; non-depleting antibodies; anti-TNF antibodies; natalizumab; anti-CD154 antibodies; soluble cytokine receptors; soluble TNF receptors; and anakinra.

In one embodiment, the additional active ingredient is rapamycin. Rapamycin may be expected to have a role in expanding the population of Treg cells in vivo.

In a further embodiment of the invention, guadecitabine is administered with one or more other additional active ingredients which also induce IDO.

Suitably the one or more additional active ingredients which also induce IDO are selected from the group consisting of cytidine analogues; histone deacetylase inhibitors; vitamin D3 analogues; interferons; toll-like receptor ligands; gonadotropine receptor signalling hormones; prostaglandine E2 analogues; IDO stabilizers; soluble CTLA4 conjugates; and glycocorticoids. More suitably, an additional active ingredient is a cytidine analogue, for example, zebularine or a pharmaceutically acceptable salt thereof. Alternatively, an additional active ingredient which induces IDO is an interferon, for example interferon gamma or interferon alpha or a pharmaceutically acceptable salt thereof. In one embodiment, the cytidine analogue is not zebularine or a pharmaceutically acceptable salt thereof.

In one embodiment, the one or more additional active ingredients which also induce IDO are selected from the group consisting of procainamide, decitabine, psammaplin A and azacytidine and pharmaceutically acceptable salts thereof.

In one embodiment, one or more additional active ingredients are other than procainamide, decitabine, psammaplin A and azacytidine and pharmaceutically acceptable salts thereof.

In a yet further embodiment, guadecitabine may be administered with two additional active ingredients such as a cytidine analogue (for example zebularine or a pharmaceutically acceptable salt thereof) and an interferon such as interferon gamma or interferon alpha or a pharmaceutically acceptable salt thereof. Suitably, the cytidine analogue is not zebularine or a pharmaceutically acceptable salt thereof.

### Pharmaceutical Compositions

Guadecitabine may be administered to the subject in a pharmaceutical composition. Therefore in one embodiment of the invention, there is provided a pharmaceutical composition comprising guadecitabine or a pharmaceutically acceptable salt thereof, and a pharmaceutically acceptable excipient, diluent or carrier. In a further embodiment, the pharmaceutical composition comprises guadecitabine or a pharmaceutically acceptable salt thereof, one or more additional active ingredients and a pharmaceutically acceptable excipient, diluent or carrier. In a yet further embodiment, the pharmaceutical composition comprises guadecitabine or a pharmaceutically acceptable salt thereof, one or more additional active ingredients which also induces IDO and a pharmaceutically acceptable excipient, diluent or carrier.

In one embodiment, an additional active ingredient in the pharmaceutical composition which also induces IDO is an interferon, for example interferon gamma or interferon alpha or a pharmaceutically acceptable salt thereof.

In another embodiment, an additional active ingredient in the pharmaceutical composition which also induces IDO is a cytidine analogue, for example zebularine or a pharmaceutically acceptable salt thereof. In one embodiment, the cytidine analogue is not zebularine or a pharmaceutically acceptable salt thereof.

In a further embodiment, the one or more additional active ingredients in the pharmaceutical composition which also induce IDO are selected from the group consisting of procainamide, decitabine, psammaplin A and azacytidine and pharmaceutically acceptable salts thereof.

In one embodiment, the one or more additional active ingredients in the pharmaceutical composition are other than procainamide, decitabine, psammaplin A and azacytidine and pharmaceutically acceptable salts thereof.

In a yet further embodiment, there may be two additional active ingredients in the pharmaceutical composition, such as a cytidine analogue (for example zebularine or a pharmaceutically acceptable salt thereof) and an interferon such as interferon gamma or interferon alpha or a pharmaceutically acceptable salt thereof. In one embodiment, the cytidine analogue is not zebularine or a pharmaceutically acceptable salt thereof.

The diluent or carrier may be an aqueous or non-aqueous solution with the purpose of diluting or carrying the medicament. The diluent or carrier may be one or more of saline, water, polyethylene glycol, propylene glycol, ethanol or oils.

The excipient may be one or more of carbohydrates, polymers, lipids and minerals. Examples of carbohydrates include lactose, sucrose, mannitol, and cyclodextrines, which are added to the pharmaceutical composition, e.g., for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate, polyethylene glycol/polyethylene oxide, polyethylene oxide/polypropylene oxide copolymers, polyvinylalcohol/polyvinylacetate of different degree of hydrolysis, and polyvinylpyrrolidone, all of different molecular weight, which are added to the pharmaceutical composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the pharmaceutical composition for reasons similar to those for polymers. Examples of minerals are talc, magnesium oxide, zinc oxide and titanium oxide, which are added to the pharmaceutical composition to obtain benefits such as reduction of liquid accumulation or advantageous pigment properties.

In a further embodiment, there is provided a pharmaceutical composition comprising guadecitabine or a pharmaceutically acceptable salt thereof and a pharmaceutically acceptable diluent or carrier for use in the prophylaxis or treatment of an immune related disease, disorder or condition. The immune related disease, disorder or condition is as defined above.

In one embodiment, the pharmaceutical composition for use in the prophylaxis or treatment of an immune related disease, disorder or condition comprises guadecitabine or a pharmaceutically acceptable salt thereof and one or more additional active ingredients. Suitably, the one or more additional active ingredients induces IDO. Example active ingredients which induce IDO are defined above.

In a further embodiment there is provided use of a pharmaceutical composition comprising guadecitabine or a pharmaceutically acceptable salt thereof for the manufacture of a medicament for the prophylaxis or treatment of an immune related disease, disorder or condition. The pharmaceutical composition may further comprise one or more additional active ingredients. Suitably, the one or more additional active ingredients induces IDO. Example active ingredients which induce IDO are defined above.

In a further embodiment there is provided a method of treating an immune related disease, disorder or condition comprising administering to a subject in need thereof a therapeutically effective amount of guadecitabine or a pharmaceutically acceptable salt thereof. The pharmaceutical composition may further comprise one or more additional active ingredients. Suitably, the one or more additional active ingredients induces IDO. Example active ingredients which induce IDO are defined above.

Administration Routes

In Vivo Administration

Guadecitabine or a pharmaceutically acceptable salt thereof, or pharmaceutical composition comprising guadecitabine or a pharmaceutically acceptable salt thereof, may be administered in vivo via any suitable route including oral, sublingual, buccal, nasal, by inhalation, parenteral i.e. by injection (including cardiac, intravenous, intra-articular, intraorgan, intramuscular, intraperitoneal, intradermal, lymphatic and subcutaneous), retrograde perfusion through cerebral venous system, via catheter into the brain parenchyma or ventricles, direct exposure or under pressure onto or through the brain or spinal tissue, or any of the cerebrospinal fluid ventricles, injections into the subarachnoid, brain cisternal, subdural or epidural spaces, via brain cisterns or lumbar puncture, intra and peri-ocular instillation including application by injection around the eye (within the eyeball and its structures and layers), the ear (including the Eustachian tube, mastoid air cells, external and internal auditory canals, tympanic membrane, middle ear, inner ear including the cochlear spiral ganglion and labyrinthine organs), as well as via enteral, bowel, rectal, vaginal, urethral or bladder cisternal. Also for in utero and perinatal indications then injections into the maternal vasculature, or through or into maternal organs including the uterus, cervix and vagina, and into embryo, foetus, neonate and allied tissues and spaces such as the amniotic sac, the umbilical cord, the umbilical artery or veins and the placenta. The preferred route may vary depending on the condition of the patient.

Suitably, the route of administration is parenteral e.g. by injection.

To treat a patient, guadecitabine or a pharmaceutically acceptable salt thereof, may be administrated at dose levels that will achieve concentrations in vivo, at the sites or locations of action, such that the concentration in vivo is for example 0.1-50 microM such as 0.1-30 microM e.g. 0.1-10 microM or other lower or higher levels that are effective, depending on which disease or disorder is to be treated.

### REFERENCES

1. http://www.cancer.gov/publications/dictionaries/cancer-drug?cdrid=691684
2. Safety and tolerability of guadecitabine (SGI-110) in patients with myelodysplastic syndrome and acute myeloid leukaemia: a multicentre, randomised, dose-escalation phase 1 study; Issa et al. The Lancet Oncology, 2015, 16(9), 1099-1110
3. http://www.astx.com/pipeline/products/clinical
4. CD137 induces proliferation and endomitosis in monocytes; Langstein et al. J. Blood, 1999, 94(9), 3161-3168

### Abbreviations

APC Antigen presenting cell
BMDC Bone marrow-derived dendritic cell
DC Dendritic cell
DNA Deoxyribonucleic acid
EGF epidermal growth factor
ELISA enzyme-linked immunosorbent assay
EPO erythropoietin
FBS fetal bovine serum
FGF fibroblast growth factor
Flt3L FMS-like tyrosine kinase 3 ligand
FVIII Factor VIII
G-CSF granulocyte colony stimulating factor
GM-CSF granulocyte macrophage colony stimulating factor
HGF hepatocyte growth factor
HPRT1 hypoxanthine phosphoribosyltransferase 1
IDO Indolamine dioxygenase
IFN interferon
Ig immunoglobulin
IGF insulin-like growth factor
IL interleukin
KGF keratinocyte growth factor
MAb monoclonal antibody
MHC major histocompatibility complex
PBMC Peripheral blood mononuclear cell
PBS Phosphate-buffered saline
PEG polyethylene glycol
RNA ribonucleic acid
SCF stem cell factor
TGF tissue growth factor
TGF-b TGF-beta
TNF tumor necrosis factor
Tregs regulatory T-cells
μ micro
VEGF vascular endothelial growth factor

### EXAMPLES

Materials and Methods

Induction of IDO1 in THP-1 Cells

THP-1 cells were harvested from appropriate number of T75 flasks. Cells were counted in a Bürker chamber, stained with 0.4% Trypan Blue. The appropriate number of cells for each experiment were transferred to a new tube and centrifuged at 1200 rpm, 5 min at room temperature (RT). The supernatant was discarded and fresh RPMI with 5% FBS were added to get a cell concentration of 2×10⁵ cells/mL. The compound was added to the five separate wells in 6-well plates such that five different final concentrations were obtained after adding the cells. A control was added to one well in the 6-well plate to which cells were added. 1×10⁶ cells per well were seeded out to achieve a total volume of 5 mL per well. The cells were incubated at 37° C. in 5% CO₂ for 96 hours. After 96 hours the assay was stopped and the cells were lysed for the isolation of RNA.

RNA Extraction and Quantification of Gene Expression by RT-qPCR

Total RNA was extracted from THP-1 cells using the RNA RNeasy Mini extraction kit (Qiagen), according to the manufacturer's instructions. A SuperScript® VILO™ RT-kit (Thermo Fisher) was used to reverse transcribe 1 μg of total RNA to cDNA and qPCR was performed. The primers used for amplification of the human IDO1 gene were 5″-TTGTTCTCATTTCGTGATGG-3″ (forward; SEQ ID No. 1) and 5″-TACTTTGATTGCAGAAGCAG-3″ (reverse; SEQ ID No. 2). The primers used for amplification of the endogenous reference gene for human HPRT1 were 5″-CTAATTATGGACAGGACTGAAC-3″ (forward; SEQ ID No. 3) and 5″-AGCAAAGAATTTATAGCCCC-3″ (reverse; SEQ ID No. 4). In brief, RT-qPCR was performed in 20 μl reaction, consisting 10 μl PowerUp SYBR Green Master Mix, 0.5 μM of each primer, 4 μl diluted cDNA for amplification of IDO1 and 1.5 μl diluted cDNA for amplification of HPRT1. An iCycler iQ real-time detection system (Stratagene, Mx3000P, La Jolla, Calif., USA) with the following thermal profile: UDG incubation at 50° C. for 2 min, then denaturation at 95° C. for 5 min, followed by 45 cycles at 94° C. for 15 s, 58° C. for 30 s, and 60° C. for 30 s, was used to perform thermocycling and real-time detection of PCR products. After amplification a melting curve analysis was performed. The RT-qPCR experiments were always run in triplicate. IDO1 was normalised to the geometric means of two HPRT1 reference gene, using the ΔCt method. Within-group comparisons were normalized to one control sample, using the ΔΔCt method.

### Example 1—Induction of IDO1 in THP-1 Cells with Guadecitabine

The procedure under “Induction of IDO1 in THP-1 cells” was followed such that the final concentrations set out in Table 1 were obtained.

TABLE 1
Final concentrations of quadecitabine in 5 mL wells

| Sample No. | Compound | Final Concentration | |
|---|---|---|---|
| 1 | DMSO (control) | 0.5% | |
| 2 | guadecitabine | 0.1 | micromolar |
| 3 | guadecitabine | 0.3 | micromolar |
| 4 | guadecitabine | 1 | micromolar |
| 5 | guadecitabine | 3 | micromolar |
| 6 | guadecitabine | 10 | micromolar |

Following RNA extraction and quantification of gene expression by RT-qPRC, as described above, induction of IDO1 gene expression using Samples 1 to 6 was investigated.

TABLE 2
Results of quadecitabine samples on IDO1 expression

| | Concentration | IDO1 expression |
|---|---|---|
| Sample No. | (micromolar) | (fold change) |
| 1 | control | 1 |
| 2 | 0.1 | 2.1 |
| 3 | 0.3 | 4.7 |
| 4 | 1 | 34 |
| 5 | 3 | 68 |
| 6 | 10 | 74 |

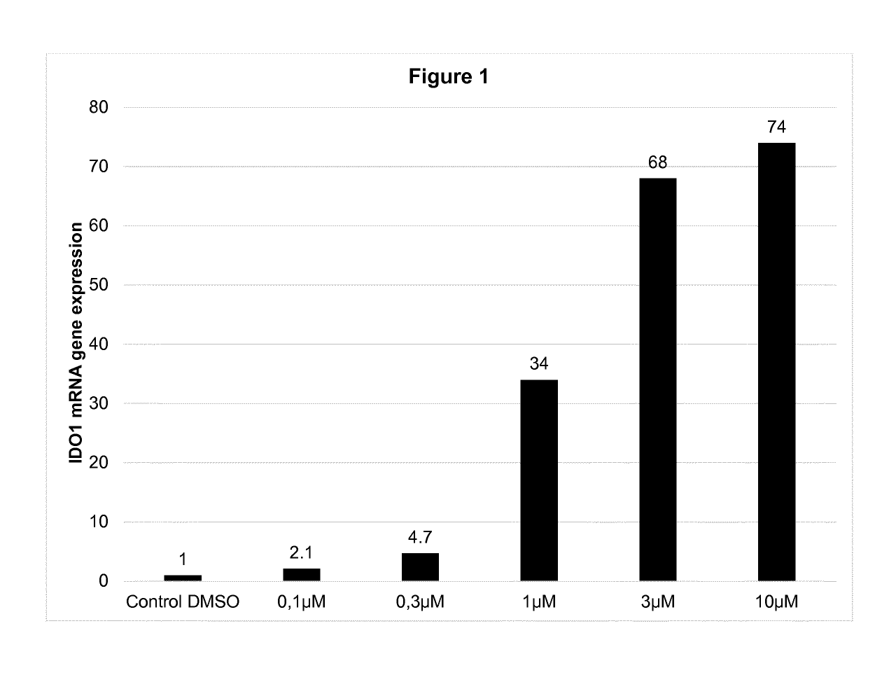

The results are also shown in FIG. 1. The results indicate that guadecitabine alone increases IDO1 gene expression by up to 74 fold. The highest level of IDO induction was observed at 10 micromolar concentration of guadecitabine.

These results suggest that guadecitabine may be used to induce IDO in cells such as antigen presenting cells in vivo or ex vivo.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications referred to herein are incorporated by reference in their entirety.

1-38. (canceled)

39. A method of treating an immune related disease, disorder or condition comprising administering to a subject in need thereof a therapeutically effective amount of guadecitabine or a pharmaceutically acceptable salt thereof.

40. A method according to claim 39 wherein the immune related disease, disorder or condition is selected from the group consisting of autoimmune diseases and disorders; immune rejection of transplants; and immune reactions to an exogenous therapeutic agent.

41. A method according to claim 40 wherein the immune related disease, disorder or condition is an autoimmune disease or disorder e.g. Achlorhydria, Acute haemorrhagic leukoencephalitis, Addison's Disease, Alopecia Areata, Anemia, Ankylosing Spondylitis, Anti-Glomerular Basement Membrane Disease, Antiphospholipid Syndrome, Aplastic Anemia, Atopic Allergy, Autoimmune Atrophic Gastritis, Autoimmune Hearing Loss, Autoimmune hemolytic anemia, Autoimmune Hepatitis, Autoimmune hypoparathyroidism, Autoimmune hypophysitis, Autoimmune Lymphoproliferative Syndrome, Autoimmune Myocarditis, Autoimmune oophoritis, Autoimmune orchitis, Autoimmune Polyendocrinopathy-Candidiasis-Ectodermal-Dystrophy, Autoimmune Polyendocrinopathy, Autoimmune Syndrome Type II, Behcet Syndrome, Celiac Disease, Chagas Disease, Chronic Active Hepatitis, Chronic Inflammatory Demyelinating Polyneuropathy, Chronic lymphocytic thyroiditis, Churg-Strauss Syndrome, Crohn's disease, Cryoglobulinemia, Cushing's Syndrome, Dermatitis Herpetiformis, Dermatomyositis, Diabetes Mellitus type 1, Diffuse Cerebral Sclerosis of Schilder, Epidermolysis Bullosa Acquisita, Erythematosis, Felty's Syndrome, Glomerulonephritis, Membranous Glomerulonephritis, Goodpasture Syndrome, Graves' Disease, Guillain-Barre Syndrome, Hamman-Rich Syndrome, Idiopathic Thrombocytopenic Purpura, Inflammatory Bowel Diseases, Insulin resistance-type B, Lambert-Eaton Myasthenic Syndrome, Lens-induced uveitis, Lichen Sclerosus et Atrophicus, Lymphopenia, Meniere's Disease, Mixed Connective Tissue Disease, Mooren's ulcer, Mucocutaneous Lymph Node Syndrome, Multiple Sclerosis, Myasthenia Gravis, Transverse Myelitis, Myocarditis, Narcolepsy, Neuromyelitis Optica, Oculovestibuloauditory Syndrome, Sympathetic Ophthalmia, Opsoclonus-Myoclonus Syndrome, Pancreatitis, Bullous Pemphigoid, Pemphigus foliaceous, Pemphigus Vulgaris, Polyarteritis Nodosa, Polymyalgia Rheumatica, Polyradiculoneuropathy, Primary biliary cirrhosis, Psoriasis, Raynaud's Disease, Reiter's Disease, Relapsing Polychondritis, Rheumatic Fever, Rheumatoid Arthritis, Sarcoidosis, Scleroderma, Sclerosing Cholangitis, Sjögren's Syndrome, Stiff-Person Syndrome, Adult-Onset Still's Disease, Takayasu Arteritis, Temporal Arteritis, Thyrotoxicosis, Type B Insulin Resistance, Ulcerative Colitis, Uveomeningoencephalitic Syndrome, Vitiligo and Wegener's Granulomatosis.

42. A method according to claim 41 wherein the autoimmune disease or disorder is selected from the group consisting of Diabetes Mellitus Type 1, Rheumatoid Arthritis, Chronic lymphocytic thyroiditis, Multiple Sclerosis and Ulcerative Colitis.

43. A method according to claim 40 wherein the immune related disease, disorder or condition is immune rejection of a transplant.

44. A method according to claim 43 wherein the transplant is an organ, tissue or cells, including normal cells or genetically modified cells.

45. A method according to claim 40 wherein the immune related disease, disorder or condition is an immune reaction to exogenous therapeutic agent.

46. A method according to claim 45 wherein the exogenous therapeutic agent is a biological drug such as FVIII, Factor IX or an antibody and the immune reaction is the raising of antibodies thereto.

47. A method according to claim 39 wherein guadecitabine is administered as a sole active ingredient.

48. A method according to claim 39 wherein guadecitabine is administered to the subject in a pharmaceutical composition.

49. A method according to claim 48 wherein the pharmaceutical composition comprises a pharmaceutically acceptable excipient, diluent or carrier.

50. A method according to claim 39 wherein guadecitabine is administered in combination with one or more additional active ingredients.

51. A method according to claim 50 wherein the one or more additional active ingredients are selected from metabolites of tryptophan; immunosuppressive reagents; aldehyde oxidase inhibitors; methotrexate; rapamycin; cyclophosphamide; antimetabolites; immunophilin-binding drugs; inhibitors of nucleotide synthesis; FTY720; lymophocyte depleting antibodies; non-depleting antibodies; anti-TNF antibodies; natalizumab; anti-CD154 antibodies; soluble cytokine receptors; soluble TNF receptors; and anakinra.

52. A method according to claim 50 wherein the one or more additional active ingredients are selected from other compounds which induces IDO such as cytidine analogues (e.g. zebularine or a pharmaceutically acceptable salt thereof); histone deacetylase inhibitors; vitamin D3 analogues; interferons (e.g. interferon gamma or interferon alpha or a pharmaceutically acceptable salt thereof); toll-like receptor ligands; gonadotropine receptor signalling hormones; prostaglandine E2 analogues; IDO stabilizers; soluble CTLA4 conjugates; and glycocorticoids.

53. A method according to claim 50 wherein the one or more additional active ingredients are selected from the group consisting of procainamide, decitabine, psammaplin A and azacytidine and pharmaceutically acceptable salts thereof.

54. A method according to claim 50 wherein the one or more additional active ingredients is rapamycin.

55. A method according to claim 39 wherein guadecitabine is administered by injection.

56. A method of preventing an immune related disease, disorder or condition comprising administering to a subject in need thereof a therapeutically effective amount of guadecitabine or a pharmaceutically acceptable salt thereof wherein the immune related disease, disorder or condition is immune rejection of transplants or immune reactions to an exogenous therapeutic agent.

57. A method according to claim 56 wherein the exogenous therapeutic agent is a biological drug such as FVIII, Factor IX or an antibody.


SEQUENCE LISTING (4 sequences)

SEQ ID NO: 1 - DNA, 20 bp, Homo sapiens
ttgttctcatttcgtgatgg

SEQ ID NO: 2 - DNA, 20 bp, Homo sapiens
tactttgattgcagaagcag

SEQ ID NO: 3 - DNA, 22 bp, Homo sapiens
ctaattatggacaggactgaac

SEQ ID NO: 4 - DNA, 20 bp, Homo sapiens
agcaaagaatttatagcccc